(19) United States
(12) Patent Application Publication
(10) Pub. No.: US 2022/0301685 A1
(43) Pub. Date: Sep. 22, 2022

(54) EAR-WEARABLE DEVICE AND SYSTEM FOR MONITORING OF AND/OR PROVIDING THERAPY TO INDIVIDUALS WITH HYPOXIC OR ANOXIC NEUROLOGICAL INJURY

(71) Applicant: Starkey Laboratories, Inc., Eden Prairie, MN (US)

(72) Inventors: Justin R. Burwinkel, Eden Prairie, MN (US); David Alan Fabry, Eden Prairie, MN (US); Gregory John Haubrich, Champlin, MN (US); Scott Thomas Klein, Minneapolis, MN (US); Adrian Lister, Victoria (CA); Paul Shriner, Hopkins, MN (US)

(21) Appl. No.: 17/698,677

(22) Filed: Mar. 18, 2022

Related U.S. Application Data
(60) Provisional application No. 63/163,100, filed on Mar. 19, 2021.

(51) Int. Cl. G16H 20/70; A61B 5/11; G06F 1/16

(52) U.S. Cl. CPC G16H 20/70; A61B 5/1124; G06F 1/163

(57) ABSTRACT

Embodiments herein relate to ear-wearable devices configured to administer therapy to individuals who have suffered anoxic or hypoxic neurological injury and/or assess recovery from such injuries and related systems and methods. In an embodiment, an ear-wearable device is included having a control circuit, a microphone, a motion sensor, and a power supply circuit, wherein the ear-wearable device is configured to initiate a therapy for a wearer of the ear-wearable device and monitor signals from the microphone and/or the motion sensor to detect execution of the therapy. In an embodiment, the ear-wearable device is configured to evaluate signals from at least one of the microphone and the motion sensor to assess recovery from an anoxic or hypoxic neurological injury. Other embodiments are also included herein.

This application claims the benefit of U.S. Provisional Application No. 63/163,100 filed Mar. 19, 2021, the content of which is herein incorporated by reference in its entirety.

### FIELD

Embodiments herein relate to ear-wearable devices configured to administer therapy to individuals who have suffered anoxic or hypoxic neurological injury and/or assess recovery from such injuries and related systems and methods.

### BACKGROUND

Cerebral hypoxia is a condition in which the brain is deprived of sufficient oxygen. Cerebral anoxia is a condition in which the brain is completely deprived of oxygen. Prolonged hypoxia or anoxia induces neuronal cell death via apoptosis, resulting in a hypoxic brain injury.

Unfortunately, hypoxic-anoxic injuries are quite common. One such type of hypoxic-anoxic injury is a stroke. It is estimated that one in four people over the age of 25 is at risk of stroke in their lifetime, and that over 15,000,000 strokes occur worldwide each year. Of these cases, roughly 15% of the victims expire shortly after the stroke and another 50% become permanently disabled. As such, stroke is a leading cause of serious long-term disability.

Approximately 85-90% of strokes are ischemic wherein a vascular blockage (i.e., infarct) occurs in a cerebral artery due to a thrombus (a clot that forms in the cerebral artery) or embolism (a clot that forms outside the brain, such as in the heart, and is then carried to the brain) within the artery. The remainder of strokes are hemorrhagic. A hemorrhagic stroke is a stroke that follows from hemorrhage or bleeding in the brain. Beyond strokes, a similar event is a transient ischemic attack (TIA). A transient ischemic attack can be caused by the same conditions that cause an ischemic stroke, but the blockage is temporary.

While every anoxic or hypoxic neurological injury is different, there is a positive correlation between the intensity and consistency of rehabilitation therapy and the magnitude of functional recovery. During rehabilitation, many individuals experience the fastest recovery during the first months after the neurological injury. This is partly attributed to intensive inpatient rehabilitation therapy, which can include multiple hours of therapy per day. However, after therapy ceases or is otherwise reduced, rehabilitation progress can quickly plateau or even regress.

### SUMMARY

Embodiments herein relate to ear-wearable devices configured to administer therapy to individuals who have suffered anoxic or hypoxic neurological injury and/or assess recovery from such injuries and related systems and methods. In a first aspect, an ear-wearable device is included having a control circuit, a microphone, a motion sensor, and a power supply circuit. The ear-wearable device can be configured to initiate a therapy for a wearer of the ear-wearable device and monitor signals from the microphone and/or the motion sensor to detect execution of the therapy.

In a second aspect, in addition to one or more of the preceding or following aspects, or in the alternative to some aspects, the ear-wearable device is configured to direct a wearer of the ear-wearable device to execute steps of the therapy.

In a third aspect, in addition to one or more of the preceding or following aspects, or in the alternative to some aspects, the ear-wearable device is configured to direct the wearer of the ear-wearable device by providing audible instructions.

In a fourth aspect, in addition to one or more of the preceding or following aspects, or in the alternative to some aspects, the therapy can include speech-language therapy.

In a fifth aspect, in addition to one or more of the preceding or following aspects, or in the alternative to some aspects, the ear-wearable device is configured to evaluate a nature or quality of a response from the ear-wearable device wearer in response to the steps of the therapy.

In a sixth aspect, in addition to one or more of the preceding or following aspects, or in the alternative to some aspects, the nature or quality of the response includes at least one of fricative stopping, liquid gliding, lisping, dysphonia, and disfluency.

In a seventh aspect, in addition to one or more of the preceding or following aspects, or in the alternative to some aspects, the ear-wearable device is configured to evaluate the ear-wearable device wearer's response to the therapy as observed by their speech outside of therapy sessions.

In an eighth aspect, in addition to one or more of the preceding or following aspects, or in the alternative to some aspects, the therapy can include swallow therapy.

In a ninth aspect, in addition to one or more of the preceding or following aspects, or in the alternative to some aspects, the swallow therapy includes a swallow protocol.

In a tenth aspect, in addition to one or more of the preceding or following aspects, or in the alternative to some aspects, the ear-wearable device is configured to use signals from the microphone and/or the motion sensor to detect head position, swallowing, and/or drinking during execution of the swallow protocol.

In an eleventh aspect, in addition to one or more of the preceding or following aspects, or in the alternative to some aspects, the ear-wearable device is configured to use signals from the microphone and/or the motion sensor to detect aspiration during execution of the swallow protocol.

In a twelfth aspect, in addition to one or more of the preceding or following aspects, or in the alternative to some aspects, the therapy can include motor skills therapy.

In a thirteenth aspect, in addition to one or more of the preceding or following aspects, or in the alternative to some aspects, the motor skills therapy can include a movement protocol.

In a fourteenth aspect, in addition to one or more of the preceding or following aspects, or in the alternative to some aspects, the motor skills therapy can include at least one of range of motion therapy, mobility training, limb movement, and virtual reality therapy.

In a fifteenth aspect, in addition to one or more of the preceding or following aspects, or in the alternative to some aspects, the therapy can include cognitive therapy.

In a sixteenth aspect, in addition to one or more of the preceding or following aspects, or in the alternative to some aspects, wherein initiating the therapy is triggered based on at least one of detection of an acoustic environment, detection of motion, and an occurrence of a specific date and/or time.

In a seventeenth aspect, in addition to one or more of the preceding or following aspects, or in the alternative to some aspects, the ear-wearable device is configured to provide an adaptive recommendation.

In an eighteenth aspect, in addition to one or more of the preceding or following aspects, or in the alternative to some aspects, the adaptive recommendation includes liquid thickening.

In a nineteenth aspect, in addition to one or more of the preceding or following aspects, or in the alternative to some aspects, the ear-wearable device is configured to track hydration of a wearer of the ear-wearable device.

In a twentieth aspect, in addition to one or more of the preceding or following aspects, or in the alternative to some aspects, the ear-wearable device is configured to send therapy instructions to an accessory device for visual presentation to the wearer of the ear-wearable device.

In a twenty-first aspect, in addition to one or more of the preceding or following aspects, or in the alternative to some aspects, the ear-wearable device is configured to receive an input from the wearer of the ear-wearable device to delay, reschedule, or cancel the therapy.

In a twenty-second aspect, an ear-wearable device is included having a control circuit, a microphone, a motion sensor, and a power supply circuit. The ear-wearable device can be configured to evaluate signals from at least one of the microphone and the motion sensor to assess recovery from an anoxic or hypoxic neurological injury.

In a twenty-third aspect, in addition to one or more of the preceding or following aspects, or in the alternative to some aspects, the ear-wearable device is configured to query the device wearer.

In a twenty-fourth aspect, in addition to one or more of the preceding or following aspects, or in the alternative to some aspects, the ear-wearable device is configured to evaluate a nature or quality of a response from the device wearer in response to the query.

In a twenty-fifth aspect, in addition to one or more of the preceding or following aspects, or in the alternative to some aspects, the ear-wearable device is configured to evaluate trends in at least one of posture, gait, sway, foot shuffling, stride symmetry, and foot fall intensity.

In a twenty-sixth aspect, in addition to one or more of the preceding or following aspects, or in the alternative to some aspects, the ear-wearable device to evaluate trends in movement speed of the device wearer.

In a twenty-seventh aspect, in addition to one or more of the preceding or following aspects, or in the alternative to some aspects, the ear-wearable device is configured to evaluate trends in movement patterns and/or activity levels of the device wearer.

In a twenty-eighth aspect, in addition to one or more of the preceding or following aspects, or in the alternative to some aspects, the ear-wearable device is configured to evaluate signals from at least one of the microphone and the motion sensor to detect patterns indicative of sequelae of an anoxic or hypoxic neurological injury.

In a twenty-ninth aspect, in addition to one or more of the preceding or following aspects, or in the alternative to some aspects, the pattern can include aspects of the ear-wearable device wearer's speech.

In a thirtieth aspect, in addition to one or more of the preceding or following aspects, or in the alternative to some aspects, the pattern can include changed pronunciation.

In a thirty-first aspect, in addition to one or more of the preceding or following aspects, or in the alternative to some aspects, the pattern can include slurred words.

In a thirty-second aspect, in addition to one or more of the preceding or following aspects, or in the alternative to some aspects, the pattern can include the clarity of the ear-wearable device wearer's speech.

In a thirty-third aspect, in addition to one or more of the preceding or following aspects, or in the alternative to some aspects, wherein clarity includes at least one of breathiness, pitch change, vowel instability, and roughness.

In a thirty-fourth aspect, in addition to one or more of the preceding or following aspects, or in the alternative to some aspects, the pattern can include long delays.

In a thirty-fifth aspect, in addition to one or more of the preceding or following aspects, or in the alternative to some aspects, the pattern can include words indicating confusion.

In a thirty-sixth aspect, in addition to one or more of the preceding or following aspects, or in the alternative to some aspects, the pattern is indicative of motor impairment.

In a thirty-seventh aspect, in addition to one or more of the preceding or following aspects, or in the alternative to some aspects, the pattern is indicative of a sudden decrease in coordination.

In a thirty-eighth aspect, in addition to one or more of the preceding or following aspects, or in the alternative to some aspects, the pattern is indicative of onset of dizziness or imbalance.

In a thirty-ninth aspect, in addition to one or more of the preceding or following aspects, or in the alternative to some aspects, the ear-wearable device is configured to detect a non-volitional body movement.

In a fortieth aspect, in addition to one or more of the preceding or following aspects, or in the alternative to some aspects, the ear-wearable device is configured to detect a non-volitional eye movement.

In a forty-first aspect, in addition to one or more of the preceding or following aspects, or in the alternative to some aspects, the ear-wearable device is configured to prompt the device wearer to look at an accessory device equipped with a camera.

In a forty-second aspect, a method of providing a therapy to an individual that has suffered an anoxic or hypoxic injury is included, the method including initiating a therapy for the individual using an ear-wearable device, and monitoring signals from a microphone and/or a motion sensor of the ear-wearable device to detect execution of the therapy.

In a forty-third aspect, in addition to one or more of the preceding or following aspects, or in the alternative to some aspects, the method can further include directing the individual to execute steps of the therapy using the ear-wearable device.

In a forty-fourth aspect, in addition to one or more of the preceding or following aspects, or in the alternative to some aspects, the method can further include directing the individual using the ear-wearable device by providing audible instructions.

In a forty-fifth aspect, in addition to one or more of the preceding or following aspects, or in the alternative to some aspects, the method can further include evaluating a nature or quality of a response from the individual in response to the therapy.

In a forty-sixth aspect, in addition to one or more of the preceding or following aspects, or in the alternative to some aspects, the nature or quality of the response includes at least one of fricative stopping, liquid gliding, lisping, dysphonia, and disfluency.

In a forty-seventh aspect, in addition to one or more of the preceding or following aspects, or in the alternative to some aspects, the method can further include observing the speech of the individual outside of therapy sessions.

In a forty-eighth aspect, in addition to one or more of the preceding or following aspects, or in the alternative to some aspects, the therapy includes speech-language therapy.

In a forty-ninth aspect, in addition to one or more of the preceding or following aspects, or in the alternative to some aspects, the therapy includes swallow therapy.

In a fiftieth aspect, in addition to one or more of the preceding or following aspects, or in the alternative to some aspects, the therapy includes motor skills therapy.

In a fifty-first aspect, in addition to one or more of the preceding or following aspects, or in the alternative to some aspects, the therapy includes cognitive therapy.

In a fifty-second aspect, in addition to one or more of the preceding or following aspects, or in the alternative to some aspects, the method can further include detecting head position, swallowing, and/or drinking during or after a therapy session.

In a fifty-third aspect, in addition to one or more of the preceding or following aspects, or in the alternative to some aspects, the method can further include detecting aspiration during or after a therapy session.

In a fifty-fourth aspect, in addition to one or more of the preceding or following aspects, or in the alternative to some aspects, wherein initiating the therapy is triggered based on at least one of detection of an acoustic environment, detection of motion, and the occurrence of a specific date and/or time.

In a fifty-fifth aspect, in addition to one or more of the preceding or following aspects, or in the alternative to some aspects, the method can further include providing an adaptive recommendation to the individual using the ear-wearable device.

In a fifty-sixth aspect, in addition to one or more of the preceding or following aspects, or in the alternative to some aspects, the method can further include tracking hydration of the individual using the ear-wearable device.

In a fifty-seventh aspect, in addition to one or more of the preceding or following aspects, or in the alternative to some aspects, the method can further include sending therapy instructions using the ear-wearable device to an accessory device for visual presentation to the individual.

In a fifty-eighth aspect, a method of monitoring recovery of an individual from an anoxic or hypoxic injury is included, the method including recording signals from at least one of a microphone and a motion sensor of an ear-wearable device, and evaluating the recorded signals to assess recovery from an anoxic or hypoxic neurological injury.

In a fifty-ninth aspect, in addition to one or more of the preceding or following aspects, or in the alternative to some aspects, the method can further include querying the individual using the ear-wearable device.

In a sixtieth aspect, in addition to one or more of the preceding or following aspects, or in the alternative to some aspects, the method can further include evaluating a nature or quality of a response from the individual in response to the query.

In a sixty-first aspect, in addition to one or more of the preceding or following aspects, or in the alternative to some aspects, the method can further include evaluating trends in at least one of posture, gait, sway, foot shuffling, stride symmetry, and foot fall intensity.

In a sixty-second aspect, in addition to one or more of the preceding or following aspects, or in the alternative to some aspects, the method can further include evaluating trends in movement speed of the device wearer.

In a sixty-third aspect, in addition to one or more of the preceding or following aspects, or in the alternative to some aspects, the method can further include evaluating trends in movement patterns and/or activity levels of the device wearer.

In a sixty-fourth aspect, in addition to one or more of the preceding or following aspects, or in the alternative to some aspects, the method can further include evaluating signals from at least one of the microphone and the motion sensor to detect patterns indicative of sequelae of an anoxic or hypoxic neurological injury.

In a sixty-fifth aspect, in addition to one or more of the preceding or following aspects, or in the alternative to some aspects, the method can further include prompting the individual to look at an accessory device equipped with a camera.

In a sixty-sixth aspect, in addition to one or more of the preceding or following aspects, or in the alternative to some aspects, the method can further include prompting the individual to read a passage.

In a sixty-seventh aspect, in addition to one or more of the preceding or following aspects, or in the alternative to some aspects, the method can further include evaluating the individual's fluency and accuracy in their ability to read the passage.

In a sixty-eighth aspect, in addition to one or more of the preceding or following aspects, or in the alternative to some aspects, the method can further include prompting the individual to tell the time shown on a clock.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of an ear-wearable device and wearer in accordance with various embodiments herein.

FIG. 2 is a graph illustrating motor function and neuroplasticity over time for an individual who has suffered a hypoxic or anoxic neurological injury.

FIG. 3 is a schematic view of an ear-wearable device in accordance with various embodiments herein.

FIG. 4 is a schematic view of the anatomy of the ear in accordance with various embodiments herein.

FIG. 5 is a schematic view of an ear-wearable device with the anatomy of the ear in accordance with various embodiments herein.

FIG. 6 is a schematic view of an accessory device in accordance with various embodiments herein.

FIG. 7 is a schematic view of an accessory device in accordance with various embodiments herein.

FIG. 8 is a schematic view of an ear-wearable device and wearer in accordance with various embodiments herein.

FIG. 9 is a schematic view of an ear-wearable device and wearer in accordance with various embodiments herein.

FIG. 10 is a schematic view of postural sway of an individual wearing a pair of ear-wearable devices in accordance with various embodiments herein.

FIG. 11 is a schematic view of an ear-wearable device and wearer in accordance with various embodiments herein.

FIG. 12 is a schematic view of an ear-wearable device and wearer in accordance with various embodiments herein.

FIG. 13 is a schematic view of an ear-wearable device system in accordance with various embodiments herein.

FIG. 14 is a schematic view of components of an ear-wearable device in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

As referenced above, hypoxic-anoxic injuries are quite common and very serious. For example, strokes (ischemic and hemorrhagic) have high rates of mortality and extremely high rates of sequalae. Also, as referenced above, there is a positive correlation between the intensity and consistency of rehabilitation and magnitude of recovery. During rehabilitation, many individuals experience the fastest recovery during the first months after the neurological injury occurs. This is partly attributed to intensive inpatient rehabilitation therapy, which can include multiple hours of therapy per day. Generally, after therapy ceases or is otherwise reduced, rehabilitation progress can quickly plateau or even regress.

However, for practical reasons inpatient therapy cannot continue indefinitely. Further, opportunities for outpatient clinical visits may become limited. As such, it is important that survivors of anoxic or hypoxic neurological injury be able to continue rehabilitation outside of clinical visits. In addition, such continued therapy must be consistent, sufficient in volume and intensity, and appropriately set to the current level of function of the individual receiving the therapy to be maximally effective.

In accordance with embodiments herein, ear-wearable devices can be used to initiate, direct, and/or manage therapies for a wearer of the ear-wearable device(s) who has suffered from an anoxic or hypoxic neurological injury. Thus, devices herein can be used to ensure that individual receive rehabilitation therapy that will drive their recovery as quickly as possible and to the highest level possible. Further, various embodiments of ear-wearable devices can be used to assess and/or track the recovery of a wearer of the device from an anoxic or hypoxic neurological injury.

Ear-wearable devices herein, including but not limited to hearing assistance devices, are uniquely valuable for assisting with the recovery of the wearer from an anoxic or hypoxic neurological injury. Because such devices are typically worn for many hours every day, they can provide an accurate measure of the true recovery state of the individual allowing for precise guidance of further therapy to appropriately set to the current level of function of the individual receiving the therapy.

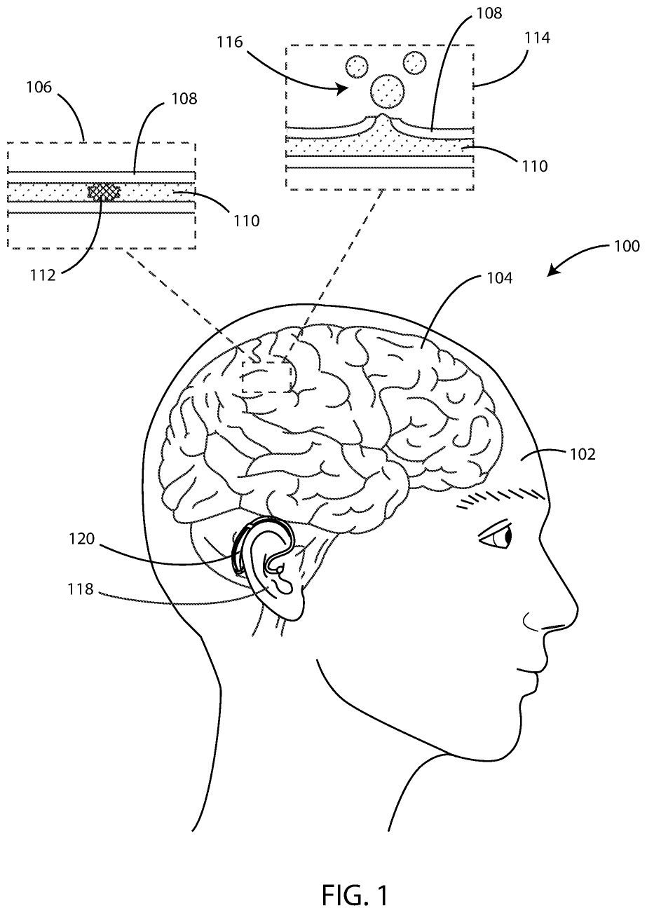

Referring now to FIG. 1, a schematic view of an ear-wearable device 120 and device wearer 100 is shown. Also shown is the head 102, brain 104, and an ear 118 of the device wearer 100. The brain 104 includes a cerebral artery 108 and blood 110 therein. FIG. 1 illustrates an ischemic stroke 106 and a hemorrhagic stroke 114. In the case of the ischemic stroke 106, the cerebral artery 108 also includes a thrombus 112. In the case of the hemorrhagic stroke 114, the cerebral artery 108 is breached leading to hemorrhage 116. It will be appreciated that while a cerebral artery 108 is depicted in FIG. 1, the illustrated concepts also apply to other portions of an individual's intracranial vascularization.

In various embodiments, an ear-wearable device 120 herein can include various components (described in greater detail below) such as a control circuit, a microphone, a motion sensor, and a power supply circuit. The ear-wearable device 120 can be configured to monitor signals from the microphone, the motion sensor, or other sensors or inputs to detect patterns indicative of sequelae of an anoxic or hypoxic neurological insult/injury. In various embodiments, the ear-wearable device 120 is configured to monitor signals from the microphone, the motion sensor, and/or other sensors or inputs to detect patterns indicative of the level of function of an individual who has suffered an anoxic or hypoxic neurological insult/injury. Exemplary patterns are described in greater detail below.

Evaluating the level of function of an individual who has suffered an anoxic or hypoxic neurological injury and identifying trends in the same over time can be clinically useful to identity appropriate therapy and/or changes in therapeutic regimens. For example, if functional recovery plateaus too quickly or does not rise sufficiently fast during periods when rapid recovery would be expected this can be a sign that current therapies are not being utilized appropriately and/or that the current therapies are not sufficient and need to be changed.

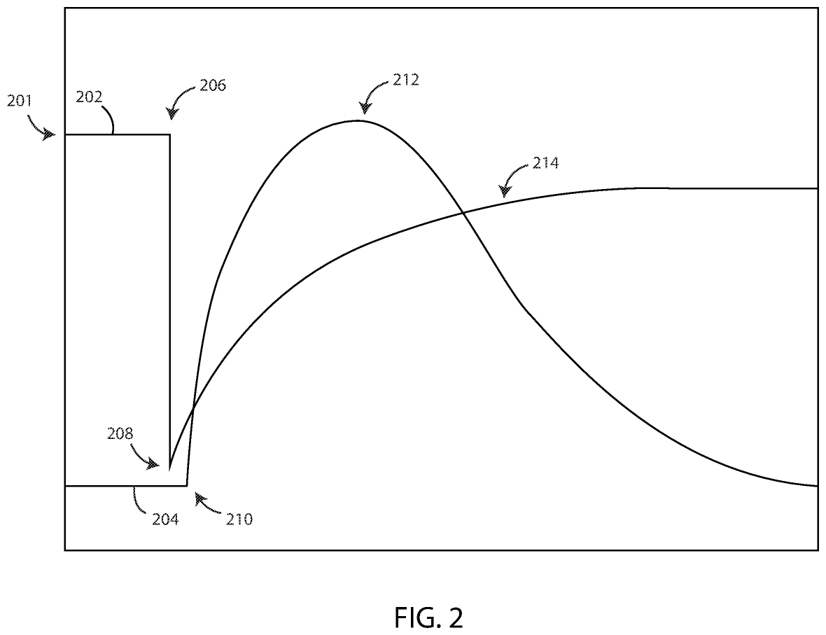

Referring now to FIG. 2, a graph is shown illustrating idealized function 202 (which could be motor function, speech function, neurological function, or the like) and neuroplasticity 204 over time for an individual who has suffered a hypoxic or anoxic neurological injury. An individual may have a preexisting level of function 201 that, upon the occurrence of a hypoxic or anoxic neurological injury 206 drops precipitously down to a low level 208. Neuroplasticity 204 may initially be relatively low, but at a time point 210 after the hypoxic or anoxic injury 206 begins to rise substantially eventually peaking 212 before gradually diminishing over time. Increases in neuroplasticity 204 can lead to a trend of increasing function 214 that eventually plateaus after a period of months. When appropriate therapy is provided, such as through the use of ear-wearable devices and systems herein, function can increase at a more rapid rate and reach a higher plateau level than would otherwise be possible.

Ear-wearable devices herein can take many different forms. In various embodiments, ear-wearable devices herein, including hearing aids and hearables (e.g., wearable earphones), can include an enclosure, such as a housing or shell, within which internal components are disposed. Components of an ear-wearable device herein can include one or more of a control circuit, digital signal processor (DSP), memory (such as non-volatile memory), power management circuitry, a data communications bus, one or more communication devices (e.g., a radio, a near-field magnetic induction device), one or more antennas, one or more microphones, a receiver/speaker, a telecoil, and various sensors as described in greater detail below. More advanced ear-wearable devices can incorporate a long-range communication device, such as a BLUETOOTH® transceiver or other type of radio frequency (RF) transceiver.

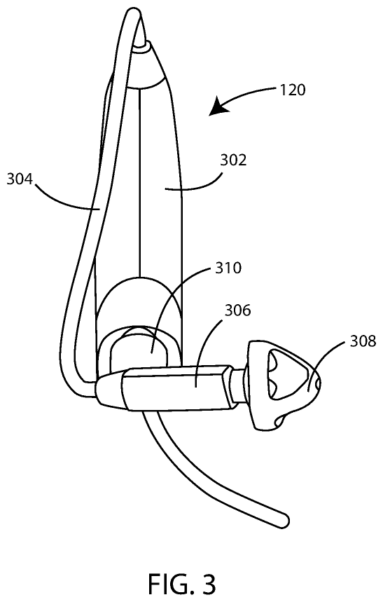

Referring now to FIG. 3, a schematic view of one example of an ear-wearable device 120 is shown in accordance with various embodiments herein. The ear-wearable device 120 can include a hearing device housing 302. The hearing device housing 302 can define a battery compartment 310 into which a battery can be disposed to provide power to the device. The ear-wearable device 120 can also include a receiver 306 adjacent to an earbud 308. The receiver 306 an include a component that converts electrical impulses into sound, such as an electroacoustic transducer, speaker, or loud speaker. Such components can be used to generate an audible stimulus in various embodiments herein. A cable 304 or connecting wire can include one or more electrical conductors and provide electrical communication between components inside of the hearing device housing 302 and components inside of the receiver 306.

The ear-wearable device 120 shown in FIG. 3 is a receiver-in-canal type device and thus the receiver is designed to be placed within the ear canal. However, it will be appreciated that many different form factors for ear-wearable devices are contemplated herein. As such, ear-wearable devices herein can include, but are not limited to, behind-the-ear (BTE), in-the ear (ITE), in-the-canal (ITC), invisible-in-canal (IIC), receiver-in-canal (MC), receiver in-the-ear (RITE), completely-in-the-canal (CIC) type hearing assistance devices, a personal sound amplifier, a cochlear implant, a bone-anchored or otherwise osseo-integrated hearing device, or the like.

Ear-wearable devices of the present disclosure can incorporate an antenna arrangement coupled to a high-frequency radio, such as a 2.4 GHz radio. The radio can conform to an IEEE 802.11 (e.g., WIFI®) or BLUETOOTH® (e.g., BLE, BLUETOOTH® 4.2 or 5.0) specification, for example. It is understood that ear-wearable devices of the present disclosure can employ other radios, such as a 900 MHz radio. Ear-wearable devices of the present disclosure can be configured to receive streaming audio (e.g., digital audio data or files) from an electronic or digital source. Representative electronic/digital sources (also referred to herein as accessory devices) include an assistive listening system, a TV streamer, a remote microphone device, a radio, a smartphone, a cell phone/entertainment device (CPED), a programming device, or other electronic device that serves as a source of digital audio data or files.

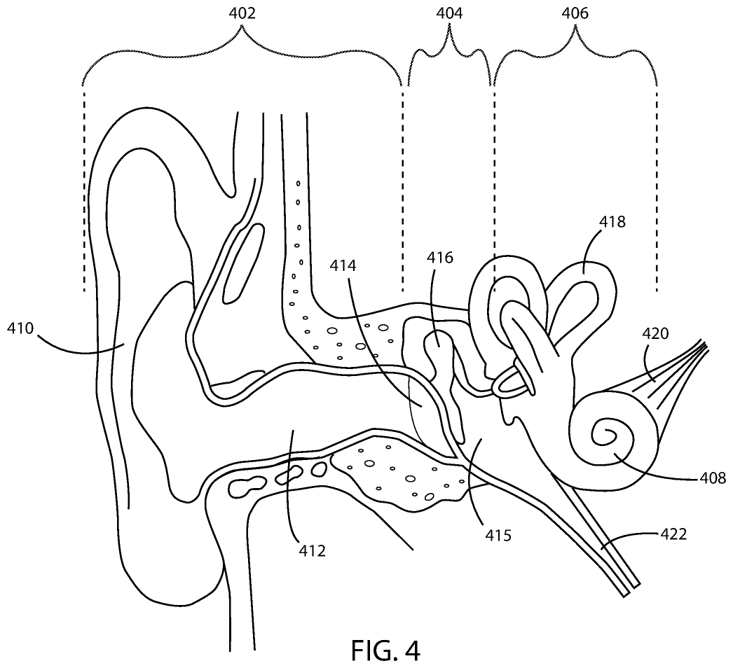

Ear-wearable devices herein can be worn on or within the ear. Referring now to FIG. 4, a partial cross-sectional view of ear anatomy is shown. The three parts of the ear anatomy are the outer ear 402, the middle ear 404 and the inner ear 406. The outer ear 402 includes the pinna 410, ear canal 412, and the tympanic membrane 414 (or eardrum). The middle ear 404 includes the tympanic cavity 415, auditory bones 416 (malleus, incus, stapes), and a portion of the facial nerve. The pharyngotympanic tube 422 is in fluid communication with the eustachian tube and helps to control pressure within the middle ear generally making it equal with ambient air pressure. The inner ear 406 includes the cochlea 408 (‘Cochlea’ means ‘snail’ in Latin; the cochlea gets its name from its distinctive coiled up shape), and the semicircular canals 418, and the auditory nerve 420.

Sound waves enter the ear canal 412 and make the tympanic membrane 414 vibrate. This action moves the tiny chain of auditory bones 416 (ossicles-malleus, incus, stapes) in the middle ear 404. The last bone in this chain contacts the membrane window of the cochlea 408 and makes the fluid in the cochlea 408 move. The fluid movement then triggers a response in the auditory nerve 420.

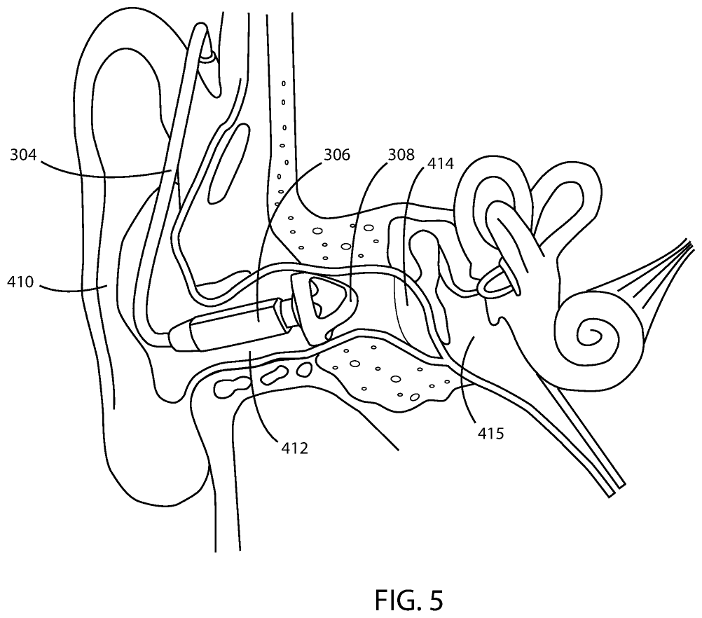

As mentioned above, the ear-wearable device 120 can be a receiver-in-canal type device and thus the receiver is designed to be placed within the ear canal. Referring now to FIG. 5, a schematic view is shown of an ear-wearable device disposed within the ear of a subject in accordance with various embodiments herein. In this view, the receiver 306 and the earbud 308 are both within the ear canal 412, but do not directly contact the tympanic membrane 414. The hearing device housing is mostly obscured in this view behind the pinna 410, but it can be seen that the cable 304 passes over the top of the pinna 410 and down to the entrance to the ear canal 412.

In various embodiments, the ear-wearable device 120 can be configured to initiate, manage, and/or guide a therapy for an individual who has suffered an anoxic or hypoxic brain injury. Therapies herein can include all types of therapies that can be beneficial for recovery of function after anoxic or hypoxic brain injury. By way of example, therapies herein can include, but are not limited to, speech-language therapy, speech therapy, articulation therapy, phonological therapy, fluency therapy, pragmatic language therapy, literacy therapy, swallow therapy, motor skills therapy, range of motion therapy, mobility training, limb movement, virtual reality therapy, cognitive rehabilitation therapy, cognitive therapy, and the like.

In some embodiments, the ear-wearable device can be configured to direct a wearer of the ear-wearable device to execute steps of the therapy. In some cases, this can take the form an instruction to a start a therapy. In other cases, it can include a series of instructions for a series of steps forming the therapy. For example, the therapy can be broken down into a series of steps for execution and instructions can be provided to the individual for each step in the series. In various embodiments, execution of the steps can be detected using sensors associated with the ear-wearable device. For example, the ear-wearable can monitor signals from the microphone and/or the motion sensor to detect execution of the therapy and/or therapy steps. In the case of a therapy involving some type of movement, signals from the motion sensor can be evaluated to detect whether or not the movement was attempted and, in some cases, how accurately it was executed. In the case of speech therapy, signals from the microphone can be evaluated to detect whether or not the speech therapy step was attempted and, in some cases, how well the individual performed the step.

In some cases, the ear-wearable device can provide feedback to the user after each step based on whether or not the ear-wearable device detects that the step has been performed and/or how well the step was performed.

In some embodiments, the ear-wearable device can be configured to evaluate a nature or quality of a response from the ear-wearable device wearer in response to the steps of the therapy. In some embodiments, the nature or quality of the response includes at least one of fricative stopping, liquid gliding, and lisping. In some embodiments, the nature or quality of the response includes the device wearer's fluency and accuracy in their ability to read a given passage or perform another task such as their ability to tell the time on a clock.

In some embodiments, such as in the case of speech therapy, the ear-wearable device can evaluate the nature and/or quality of the device wearer's speech in order to evaluate the device wearer's response to the therapy. In some embodiments, the ear-wearable device can be configured to evaluate the ear-wearable device wearer's response to the therapy as observed by their speech during therapy sessions. However, in some embodiments, the ear-wearable device can be configured to evaluate the ear-wearable device wearer's response to the therapy as observed by their speech outside of time windows associated with therapy sessions.

In some embodiments, a therapy herein can include a swallow therapy that can include a swallow protocol. Swallowing leads to both characteristic movements and characteristic sounds. In various embodiments, the ear-wearable device can be configured to use signals from the microphone and/or the motion sensor to detect head position, swallowing (such as swallowing sounds or swallowing motion), and/or drinking (such as drinking sounds or drinking motion) during execution of the swallow protocol.

Aspiration refers to fluids or other materials from the mouth entering the lungs. Aspiration can lead to problems such as aspiration pneumonia, which is a type of lung infection resulting from a relatively large amount of material from the stomach or mouth entering the lungs. Aspiration typically includes specific sounds. In some embodiments, the ear-wearable device can be configured to use signals from sensors such as a microphone and/or a motion sensor to detect possible aspiration occurring during execution of the swallow protocol.

Aspiration may indicate that the individual is experiencing difficulty swallowing. In some embodiments, the ear-wearable device can be configured to provide an adaptive recommendation. The adaptive recommendation may take on many different forms. In the specific example of detecting aspiration, the adaptive recommendation may include a recommendation to thicken liquids that are being swallowed. The recommendation can be provided to the device wearer and/or to a care provider in the form of a recommendation, alert, or notification and can be presented audibly, visually, or in other forms.

Initiation of therapies by the ear-wearable device herein can, in some cases, be triggered according to a predetermined schedule. However, in some embodiments, the therapy may be triggered based on detecting a circumstance or condition that may correspond with an ideal time for the device wearer to execute a therapy. For example, in some cases a therapy may be ideally conducted in a room that is relatively quiet. As such, in some cases, the ear-wearable device can detect that there is little ambient sound or an amount of sound falling below a threshold value and initiate therapy. The sound levels can be evaluated as an average value over a period of time in order to reduce the influence of outlier sounds. In some cases, the ear-wearable device can detect either motion or the absence of certain types of motion and initiate therapy. As such, in some embodiments, initiation of therapies herein can be based on at least one of detection of a particular acoustic environment, detection of motion, and an occurrence of a specific date and/or time.

In some embodiments, the ear-wearable device can receive input from the user such as facilitate the device wearer rescheduling their therapy session for a later time. For example, the device wearer could be presently busy with other activity and not want the therapy session to be initiated at that time. As such, the user can provide an input such as a verbal command which could be received through a microphone of the ear-wearable device, a device tap, an input through an accessory device such as a smartphone or smart watch which causes the initiation of a delay or rescheduling of the therapy. For example, it can delay the therapy by a specific time period, such as delaying by a period of minutes or hours, or it can cancel the current therapy session entirely.

Instructions/directions to the device wearer can take on various forms. In some embodiments, the instructions can be provided audibly through the ear-wearable device itself and/or an accessory device. In some embodiments the instructions can be provided visually through an accessory device. For example, in some cases the ear-wearable device can be configured to send therapy instructions to an accessory device for visual presentation to the wearer of the ear-wearable device. In some embodiments, the instructions can be provided haptically. In some embodiments, the instructions can be provided through more than one channel, simultaneously or otherwise, such as providing two or more of audible, tactile, and visual instructions.

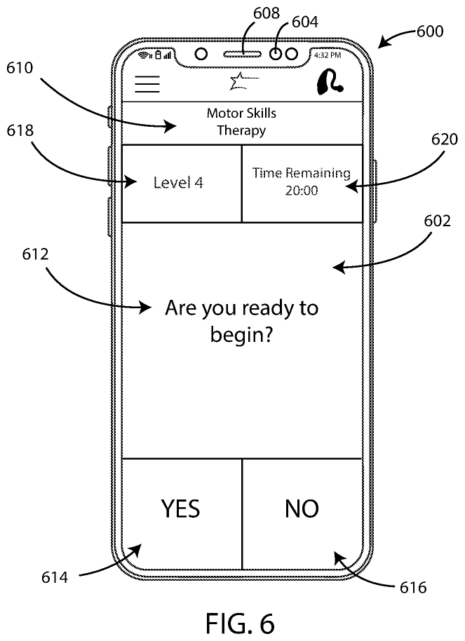

Referring now to FIG. 6, a schematic view of an accessory device 600 is shown in accordance with various embodiments herein. The accessory device can include display screen 602, camera 604, speaker 608, therapy type indication 610, query 612, first user input button 614, and second user input button 616. Various pieces of information about the therapy can also be displayed. For example, in some embodiments the level 618 or intensity of the therapy can be displayed. In some embodiments the amount of time remaining 620 for the therapy can be displayed.

In this example, the ear-wearable device 120, via the accessory device 600, can be configured to query the device wearer 100 if they are ready to start a therapy. For example, in some cases the query could be as simple as “Are you ready to begin?” as shown in FIG. 6. In some cases, the query can be more complex. The individual can then respond by interfacing with one of the user input buttons or simply speaking their answer.

It will be appreciated that instructions and/or queries herein can take on many different forms. In some embodiments, the query can be visual, aural, tactile or the like. In some embodiments, the query can request device wearer feedback or input (such as could be provided through a button press, an oral response, a movement, etc.). In some embodiments, the query can take the form of a question regarding how the device wearer 100 is feeling or what they are experiencing. In some embodiments, the query can relate to whether they are experiencing weakness. In some embodiments, the query can take the form of a question which requires a degree of cognition in order to answer, such as a math question, a verbal question, a question about their personal information (such as one for which the answer is already known by the system such as a date and/or place of birth, a current address, a home phone number, etc.), or the like. In some embodiments, the query can be a prompt for the user to read a passage. In some embodiments, the system may prescribe the content the user is to read. In another embodiment, the user may be instructed to read whatever material is available to them (no bounds) or a specific type of material that is available them (e.g., a newspaper clipping) such that the general difficulty level of the passages is known to the system even if the exact content is not. In some cases, the query can target a response which tests a specific function/area of the brain (e.g., a specific language ability like differentiating phonological or semantic differences between test stimuli). In some cases, there can be a single query. In some cases, there can be multiple queries.

In some embodiments, the ear-wearable device 120 can be configured to evaluate a nature or quality of a response from the device wearer 100 in response to the query. For example, in the context of a question, the system can evaluate whether the answer to the question suggests they are feeling ill or experiencing a symptom of a neurological injury. As another example, the system can evaluate whether the answer to a question is correct or not. As another example, the system can evaluate the amount of time taken for the device wearer to answer a question. Of course, in some cases a device wearer may simply not respond to a query. In some embodiments, the system can interpret the lack of a response as being indicative of sequelae of an anoxic or hypoxic neurological injury. However, in other embodiments, the system can be configured so as to not interpret the lack of a response that way. In some embodiments, the system can be configured to allow the user to cease or skip further therapy.

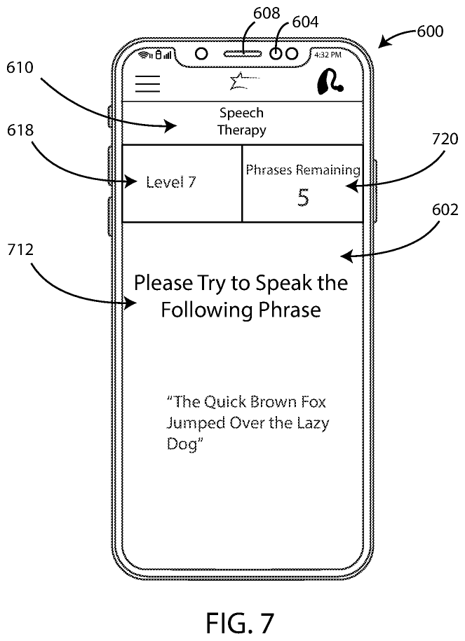

In some embodiments, a therapy instruction can specifically take the form of a request or prompt for the device wearer 100 to do or say something. Referring now to FIG. 7, a schematic view of an accessory device 600 is shown in accordance with various embodiments herein. FIG. 7 is generally similar to FIG. 6. However, in this case, an instruction 712 or direction associated with a therapy is presented to the device wearer 100. In addition, an indication of the remaining number of therapy steps 720 is also provided by way of the accessory device 600.

In this specific example, the instruction 712 directs the device wearer to speak the phrase “The quick brown fox jumped over the lazy dog.” After providing the instruction, the ear-wearable device can monitor for the execution of the instruction (e.g., the device wearer speaking the phrase) as well as the nature and quality of the execution (e.g., the accuracy of speaking the phrase, the pronunciation of the phrase, the time taken to speak the phrase, etc.). It will be appreciated that the nature of the instruction is directly related to the type of therapy being administered. For example, in some embodiments, the instruction can take the form of things like “please lift your arm”, “touch your right ear”, etc. In some embodiments, instruction 712 can include a prompt to execute a specific movement protocol.

Beyond initiating, guiding, and/or monitoring therapy, the ear-wearable device can monitor, track or assess the recovery of the individual who has suffered an anoxic or hypoxic neurological injury. For example, the ear-wearable device can monitor, track or assess the functional status of the individual who has suffered an anoxic or hypoxic neurological injury. This can be done in various ways. In some embodiments, the ear-wearable device can be configured to evaluate signals from at least one of the microphone and the motion sensor to assess recovery from an anoxic or hypoxic neurological injury.

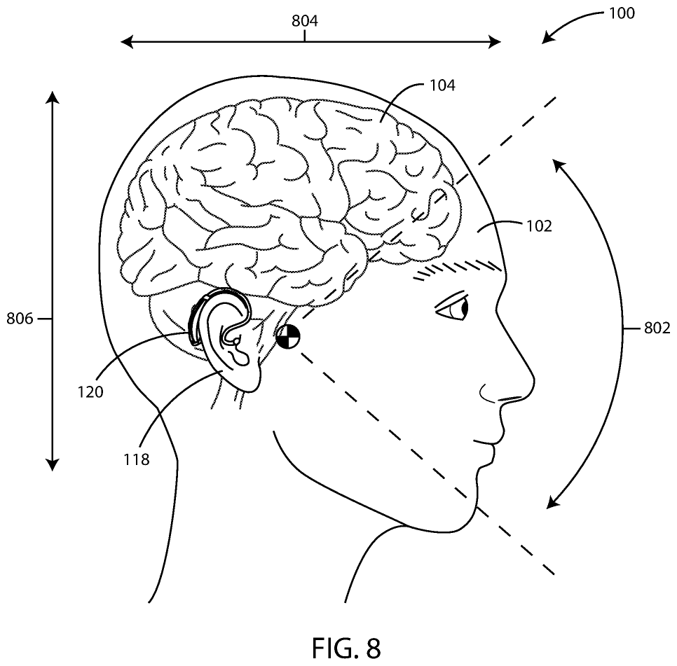

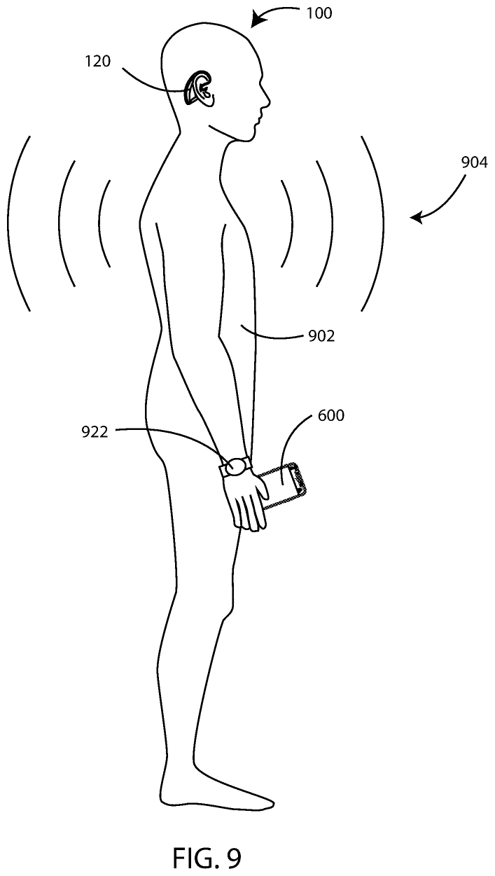

In monitoring the recovery and/or functional status of the ear-wearable device, movement of the individual wearing the ear-wearable device can be tracked. Referring now to FIG. 8, a schematic view is shown of an ear-wearable device 120 and device wearer 100. It will be appreciated that many different aspects of the device wearer 100 can be tracked with devices and systems herein. For example, in some embodiments the ear-wearable device 120 can include a motion sensor (described in greater detail below) and can sense movement of the device wearer 100. For example, with respect to both the head 102 of the device wearer 100 and other parts of their body, the system or device can sense rotational movement 802 (within multiple planes), front to back movement 804, up and down movement 806, pitch, roll, yaw, twisting motions, and the like. Referring now to FIG. 9, a schematic view of an ear-wearable device 120 and device wearer 100 is shown along with the body 902 of the device wearer 100. Other types of movement that can be sensed include body sway 904 (and in some scenarios can also include head sway). In some embodiments, such movements can be given an activity classification by the system.

As referenced above, many different patterns can be detected by the ear-wearable device and/or the ear-wearable device system in order to detect the recovery state of an individual who has suffered a neurological injury. For example, in some embodiments, the ear-wearable device 120 can detect a pattern that is indicative of motor impairment. In various embodiments, the ear-wearable device 120 can detect a pattern is indicative of one or more of gait ataxia, difficulty standing or walking, or a sudden decrease in motor coordination. In various embodiments, the ear-wearable device 120 can detect a pattern is indicative of onset of dizziness or imbalance. In various embodiments, the ear-wearable device 120 wherein the ear-wearable device 120 is configured to detect a non-volitional body movement.

In some embodiments, patterns herein can relate to the individual's gait and which can be detected with a motion sensor herein including, for example, gait speed, step distance, bilateral step comparison, footfall magnitude, and the like.

FIG. 9 also shows a wearable device 922, which could be a smartwatch, a cardiac sensor/monitor, an oxygen sensor, or the like. FIG. 9 also shows an accessory device 600, which could be a smartphone, a tablet device, a general computing device, or the like. In some embodiments, the wearable device 922 and the accessory device 600 can both be part of the ear-wearable device system. In some embodiments, the wearable device 922 and the accessory device 600 can include sensors, such as any of the sensors described herein below. In some embodiments, they can send data to the ear-wearable device. In some embodiments, they can receive data from the ear-wearable device. In some embodiments, data obtained from one or more of the ear-wearable device 120, wearable device 922, and accessory device 600 can be used to assist in detecting indicators of possible ipsilesional limb ataxia.

In some cases, the system can include a motion sensor to pick up essential tremors (unintentional, somewhat rhythmic, muscle movement involving to-and-fro movements or oscillations of one or more parts of the body) of the wearer. By way of example, some individual recovering from a stroke suffer uncontrollable shaking that can be identified within the signals of various sensors herein including motion sensors.

In some embodiments, the system can detect dysphagia (swallowing difficulty), swallowing apraxia, buccofacial apraxia, and/or aspiration. Dysphagia, swallowing apraxia, buccofacial apraxia, and/or aspiration can be an indication of ischemic strokes or TIAs. The devices or system herein can detect dysphagia, swallowing apraxia, buccofacial apraxia, and/or aspiration using data from various sensors. By way of example, dysphagia, swallowing apraxia, buccofacial apraxia, and/or aspiration can be detected by detecting a signature or pattern in microphone data and/or motion sensor data. In some embodiments, detection of a signature or pattern of dysphagia, swallowing apraxia, buccofacial apraxia, and/or aspiration can be used herein as indicative of a state of recovery from a neurological injury of a device wearer.

In various embodiments, recovery from an anoxic or hypoxic neurological injury can be assessed by evaluating the gait of the wearer of an ear-wearable device herein. Thus, in accordance with various embodiments herein, the device wearer's gait and/or balance can be evaluated. Gait analysis can include the evaluation of body movements, body mechanics, and the activity of the muscles during human motion generally and, in particular, during movements such as walking or running. Specific parameters of gait analysis can include, but are not limited to, step length (right, left), stride length, stride length to lower extremity length ratio, horizontal dimension of stride, base of support, stride cycle element analysis, frequency (cadence), speed, dynamic base, progression line, foot angle, hip angle, and the like.

In accordance with embodiments herein, one or more of an IMU unit and a microphone herein can detect movements and/or vibrations in order to identify what stage of the stride cycle the device wearer is currently in along with frequencies and time associated with the same. The biomechanics associated with such feet/ground contact results in characteristic acoustic and inertial changes that can be detected by one or more microphones and/or accelerometers (or other component) of an IMU, either alone or in combination. In some embodiments, characteristics of feet/ground contact can include a signal intensity. In some embodiments, characteristics of feet/ground contact can include a time interval. In some embodiments, characteristics of feet/ground contact can include an angular position of one or more parts of the body. For example, as one leg swings forward support by the other leg involves a characteristic vertical motion at a relatively low frequency that can be detected by a component of the IMU.

Characteristic medio-lateral axis movement can also be detected by the IMU during different phases of the stride cycle allowing each point to be identified along with timing of the same. By way of example, a limping gait can be reflected as unequal swing durations between each leg and this type of abnormal gait can be detected by the system. As another example, a shuffling-type gait can be reflected as a measurable variability in the timing of the different phases of the stride cycle that crosses a threshold value of variability (the threshold value either being pre-selected and programmed into the device or reflecting a statistical measure of deviation from an average for the specific individual as calculated over a look-back period or during a previous calibration period or event). A shuffling-type gait can also be detected using acoustic information obtained from one or more microphones.

In addition, by combining the information content provided by signals associated with directional movement in the horizontal plane (as can be measured by the IMU, microphone, or geolocation-type sensors) with that provided by stride cycle analysis as detailed above, aspects such as step length (right, left) and stride length can be calculated. These values can also be subjected to analysis to determine various statistics, e.g., absolute values (average right step length, average left step length, average stride length) as well as ratios of the same (ratio of average right step length vs. average left step length) and measures of variability in the same, and the like. In various embodiments, the system can be configured to evaluate these measures by comparison with a threshold value or confidence interval for significance, wherein the threshold value can be pre-selected and programmed into the device or reflect a statistical measure of deviation based on a statistically measured value for the specific individual as calculated over a look-back period or during a previous calibration period or event.

In various embodiments herein, the system can further evaluate progression line of locomotion as a component of gait analysis. Progression line reflects deviations from a predominant direction of locomotion that may occur non-volitionally. By way of example, when walking in a particular direction, movements along a medio-lateral axis (as can be measured by the IMU or other sensors herein) can contribute to variation in the progression line of the device wearer. One approach to measuring variation in progression line is to take the absolute magnitude of movement along a medio-lateral axis and divide by a fixed distance of travel in the predominant movement direction (walking/running direction). Various approaches can be used for measuring variation in progression line.

Postural control consists of both postural steadiness associated with the ability to maintain balance during quiet standing and postural stability that is associated with the response to applied external stimuli and volitional movements. Postural sway describes horizontal movements of an individual around the subject's center of gravity (COG) over their base of support. Aspects of postural sway can be observed during quiet standing as well as during volitions movements such as walking. Postural sway can include anterior-posterior axis motion, medial-lateral axis motion, and combinations thereof in the horizontal plane. Postural sway can be sensed and/or tracked in accordance with various embodiments herein.

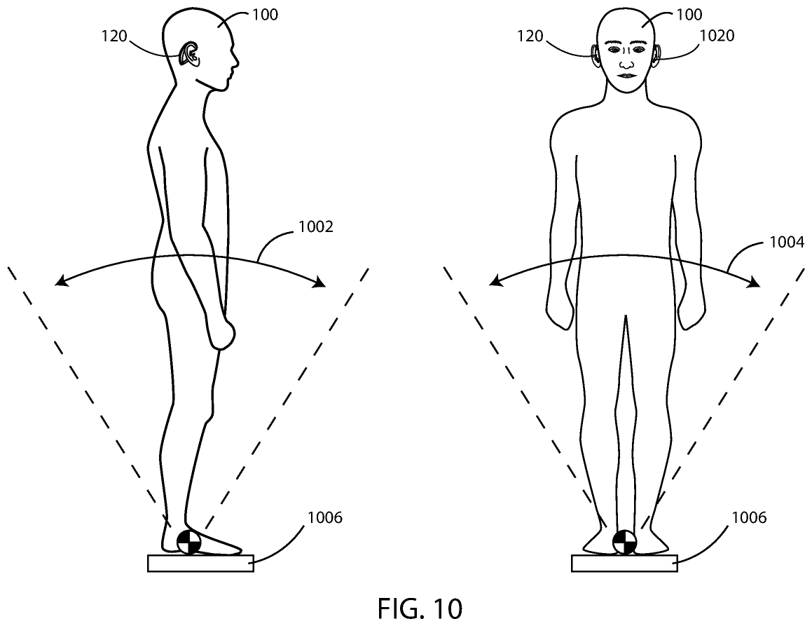

Referring now to FIG. 10, a schematic view is shown of postural sway of a device wearer 100 wearing a pair of ear-wearable devices 120, 1020 in accordance with various embodiments herein. Postural sway can include anterior-posterior axis motion 1002, medial-lateral axis motion 1004, as well as combinations thereof. It will be appreciated that movement contributing to sway can include movement initiated at any point of the body including at the level of the feet and ankles, movement at the level of the knees, hips, back, neck, head, and the like.

Parameters of postural sway that can be sensed herein can include, but are not limited to, sway size (distance), sway velocity, sway frequency, slow sway components (0.1 to 0.5 Hz), fast sway components (0.5 to 1 Hz), and the like. By way of example, an IMU unit herein (such as associated with the ear-worn device) can detect movements and/or vibrations in order to identify what stage of the stride cycle the device wearer is currently in along with frequencies and time associated with the same. The vestibulocollic reflex (VCR) acts to stabilize the head (such as by acting upon muscles in the neck to counter movement sensed by otoliths or semicircular canals), but individuals with abnormal sway may still exhibit a measurable sway of the head. While not intending to be bound by theory, detection of sway in the head is highly probative of disfunction impacting balance and stability, which can be related to neurological injury and/or functional recovery from the same.

In some embodiments, detection of sway can be performed automatically by the system without the volitional participation of the device wearer. In some cases, the system can measure sway regardless of the current activity of the device wearer. However, in other embodiments, the system can wait until e.g., a standing state is detected (which could occur as the device wearer is standing in line or otherwise standing, but not moving within the horizontal plane), a walking state is detected, or the like.

In some embodiments, the device can provide instructions for the device wearer to follow, such as “please stand still” (explicitly or implicitly) provided. For example, instructions can be provided directly from the ear-worn device through audible or tactile channels. In some embodiments, instructions can be provided from an external device through one or more of an audible, visual, or tactile modality.

In some cases, the movements to track, measure, or monitor postural sway can be performed with sensors associated with the ear-worn devices alone. However, in other cases, sensors associated with other devices can be used in addition to, or in place of, the sensors associated with the ear-worn devices (in which case signals from a different device relevant to postural sway can be sent to the ear-worn devices or a different device). By way of example, in some embodiments, a pressure-plate device 1006 can be used to identify movement or weight bearing/transfer related to postural sway. The pressure-plate device 1006 can include one or more load cells or other types of related sensors such as pressure-sensors and the like to detect sway and aspects thereof. In some embodiments, elements of a pressure-plate device 1006 can be embedded within a surface, such as a floor.

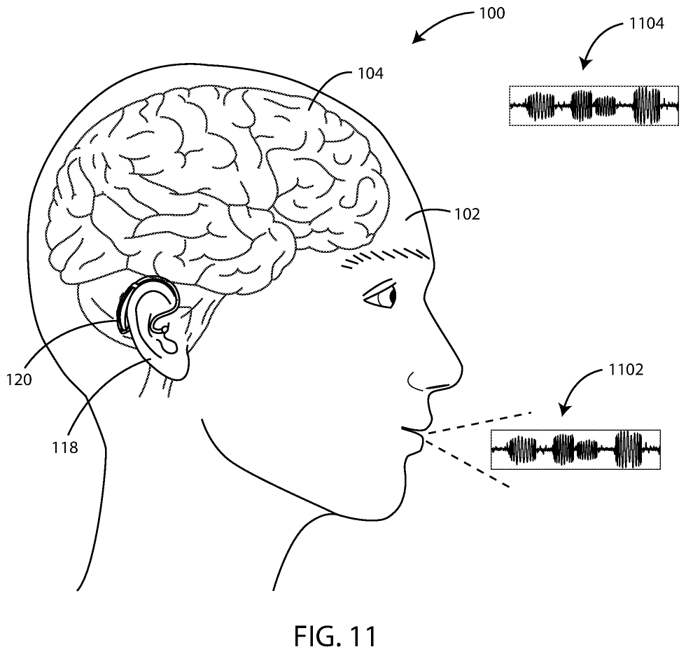

In various embodiments herein, the state of recovery and/or functional state of the individual who has suffered an anoxic and/or hypoxic neurological injury can be assessed by evaluating the speech of the individual. Referring now to FIG. 11, a schematic view of an ear-wearable device 120 and device wearer 100 is shown in accordance with various embodiments herein. In this case, various speech or noise within the environment of the device wearer 100 can be detected. For example, the ear-wearable device 120 can detect speech such as device wearer speech 1102 as well as third party speech 1104 or ambient noise.

It can be appreciated that while the ear-wearable device 120 should evaluate speech of the device wearer to evaluate the status of recovery from neurological injury of the device wearer 100, it should typically not use the speech of a third party to make such an evaluation. As such, in various embodiments herein, the device or system can distinguish between speech or sounds associated with the device wearer 100 from speech or sounds associated with a third party. Processing to distinguish between the two can be executed by any devices of the system individually or by a combination of devices of the system. In some embodiments, data used for distinguishing can be exported from an ear-wearable device or devices to one or more separate devices for processing.

Distinguishing between speech or sounds associated with the device wearer 100 and speech or sounds associated with a third party can be performed in various ways. In some embodiments, this can be performed through signal analysis of the signals generated from the microphone(s). For example, in some embodiments, this can be done by filtering out frequencies of sound that are not associated with speech of the device-wearer. In some embodiments, such as where there are two or more microphones (on the same ear-wearable device or on different ear-wearable devices) this can be done through spatial localization of the origin of the speech or other sounds and filtering out, spectrally subtracting, or otherwise discarding sounds that do not have an origin within the device wearer 100. In some embodiments, such as where there are two or more ear-worn devices, own-voice detection can be performed and/or enhanced through correlation or matching of intensity levels and or timing, and/or spectral shaping approaches.

In some cases, the system can include a bone conduction microphone in order to preferentially pick up the voice of the device wearer. In some cases, the system can include a directional microphone that is configured to preferentially pick up the voice of the device wearer. In some cases, the system can include an intracanal microphone (a microphone configured to be disposed within the ear-canal of the device wearer) to preferentially pick up the voice of the device wearer. In some cases, the system can include a motion sensor (e.g., an accelerometer configured to be on or about the head of the wearer) to preferentially pick up skull vibrations associated with the vocal productions of the device wearer.

In some cases, an adaptive filtering approach can be used. By way of example, a desired signal for an adaptive filter can be taken from a first microphone and the input signal to the adaptive filter is taken from the second microphone. If the hearing aid wearer is talking, the adaptive filter models the relative transfer function between the microphones. Own-voice detection can be performed by comparing the power of an error signal produced by the adaptive filter to the power of the signal from the standard microphone and/or looking at the peak strength in the impulse response of the filter. The amplitude of the impulse response should be in a certain range in order to be valid for the own voice. If the user's own voice is present, the power of the error signal will be much less than the power of the signal from the standard microphone, and the impulse response has a strong peak with an amplitude above a threshold. In the presence of the user's own voice, the largest coefficient of the adaptive filter is expected to be within a particular range. Sound from other noise sources results in a smaller difference between the power of the error signal and the power of the signal from the standard microphone, and a small impulse response of the filter with no distinctive peak. Further aspects of this approach are described in U.S. Pat. No. 9,219,964, the content of which is herein incorporated by reference.

In another approach, system uses a set of signals from a number of microphones. For example, a first microphone can produce a first output signal A from a filter and a second microphone can produce a second output signal B from a filter. The apparatus includes a first directional filter adapted to receive the first output signal A and produce a first directional output signal. A digital signal processor is adapted to receive signals representative of the sounds from the user's mouth from at least one or more of the first and second microphones and to detect at least an average fundamental frequency of voice (pitch output) F₀. A voice detection circuit is adapted to receive the second output signal B and the pitch output F₀ and to produce an own voice detection trigger T. The apparatus further includes a mismatch filter adapted to receive and process the second output signal B, the own voice detection trigger T, and an error signal E, where the error signal E is a difference between the first output signal A and an output O of the mismatch filter. A second directional filter is adapted to receive the matched output O and produce a second directional output signal. A first summing circuit is adapted to receive the first directional output signal and the second directional output signal and to provide a summed directional output signal (D). In use, at least the first microphone and the second microphone are in relatively constant spatial position with respect to the user's mouth, according to various embodiments. Further aspects of this approach are described in U.S. Pat. No. 9,210,518, the content of which is herein incorporated by reference.

In various embodiments, the ear-wearable device 120 can detect a pattern based on the content of the ear-wearable device 120 wearer's speech utterances. In some cases, the content can include the words that are spoken by the device wearer. In some cases, the content can include the sounds (i.e., phonemes) or sound patterns other than words that are uttered by the device wearer. In some cases, the content can include both the words and other sounds or sound patterns.

Signals reflecting the ear-wearable device wearer's speech utterances can be transcribed into words or phonemes (i.e., speech recognition) in various ways. In some embodiments, a speech-to-text module can be included within the system herein or can be accessed as part of a remote system such as an API. For example, one such speech-to-text API is the Google Cloud Speech-to-Text API, wherein files/data representing speech can be submitted and text can be retrieved. Another is the speech service API from Microsoft Azure Cognitive Speech Services.

In some embodiments, the system can evaluate the number or classification of words or phonemes reflecting confusion as uttered by the ear-wearable device wearer can be tracked. Words of confusion can include “what?” “who?”, “why?”, “when?”, “where?”, “uh?”, as well as others. In some embodiments, a value reflecting the number of words of confusion uttered per unit time (such as per minute, etc.) can be calculated. If this value changes substantially for an individual over a baseline value (such as by greater then 5, 10, 15, 20, 30, 50, 75, 100, 200 percent or more, or an amount falling within a range between any of the foregoing), then that can be taken as a pattern indicative of a particular state of recovery from a neurological injury. In other embodiments, if such values cross threshold amounts, then that can be taken as a pattern indicative of a particular state of recovery from a neurological injury.

In some embodiments, the system can use the transcription data (e.g., speech-to-text output data) associated with the device wearer's speech in order to verify whether the device wearer is answering questions correctly. For example, the system could provide a prompt, such as “What day is it?” and then wait for an answer from the device wearer. A series of similar questions could be asked and then the system could determine a score based on the number of correct answers. This could be done periodically over time. If this value is substantially reduced for an individual over a baseline value or if the score crosses a threshold amount, then that can be taken as a pattern indicative of a state of recovery from a neurological injury.

In some embodiments, the system can present images of objects on a display screen and ask the user to identify the objects and the results can be scored. In some embodiments, the system can measure the amount of time required for the device wearer to answer an open-ended question such as describing their environment. In some embodiments, the system can administer a memory test such as providing information for the device wearer to remember and then asking them to recall the provided information. In some embodiments, the system can ask questions such as “tell me words that begin with the letter ‘E’” and then score the answers, such as by counting the number of words generated by the device wearer that correctly begin with the letter “E”. In some embodiments, the system could ask a question reflecting common knowledge such as “Tell me the ingredients you might put on a pizza” and then score the results, such as by the total number of items stated by the device wearer. Any of these queries (or others) can be repeated periodically. The resulting score or value change over time can be taken as a pattern indicative of a state of recovery from a neurological injury. In some embodiments herein, queries can be generated and/or delivered by a component of the ear-wearable device system. However, in some embodiments, a third party may be generating and/or delivering the queries and a component of the ear-wearable device system can identify that a query is being delivered and monitor for a response.

It will be appreciated that speech patterns that can be evaluated herein to detect a state of recovery from an anoxic or hypoxic neurological injury can include various features. In some embodiments, the speech pattern can include long delays. For example, the system can track the amount of time between words, between spoken sentences, and or the amount of time between a query and a response. In some cases, an average delay can be calculated. In some embodiments, a time ratio of delay to spoken word content time can be calculated for a given time period (e.g., total delay time per minute/total spoken word content time per minute). If such delays (in the absolute, as an average or other statistical measure, as a ratio, etc.) increase significantly over a baseline value (such as by greater then 5, 10, 15, 20, 30, 50, 75, 100, 200 percent or more, or an amount falling within a range between any of the foregoing), then that can be taken as a pattern indicative of a particular state of recovery from a neurological injury. In other embodiments, if such values cross threshold amounts, then that can be taken as a pattern indicative of a particular state of recovery from a neurological injury. In some embodiments, the amount of time that a particular speech phoneme is sustained may be atypically long or short.

In various embodiments, the speech pattern can include the clarity, breathiness, pitch change, vowel instability, and/or roughness of the ear-wearable device wearer's speech. In various embodiments, the speech pattern can include slurred utterances. In various embodiments, the speech pattern can include strained utterances. In various embodiments, the speech pattern can include quiet utterances. In various embodiments, the speech pattern can include raspy utterances. In various embodiments, the speech pattern can include changed pronunciation of words.

In some embodiments, speech patterns herein indicative of a state of recovery from a neurological injury can include changes in speech complexity (e.g., semantic complexity, grammatical incompleteness, etc.) or fluency (e.g., atypical pause patterns) may be a signs of aphasia, dysarthria, dyspraxia or other speech-language processes associated with a stroke.

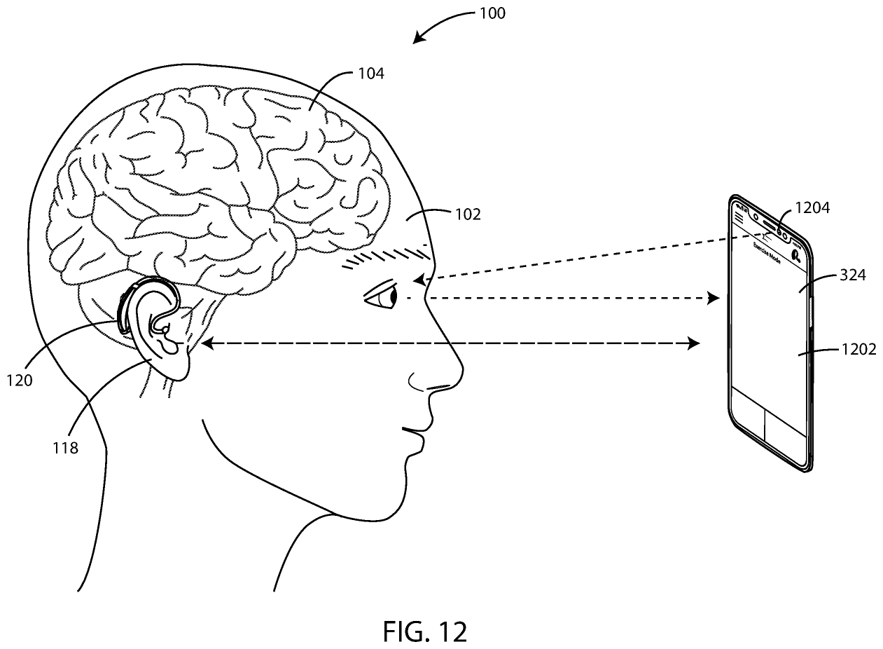

Referring now to FIG. 12, a schematic view of an ear-wearable device 120 and device wearer 100 is shown in accordance with various embodiments herein. The head 102 of the device wearer 100 is facing towards an accessory device 600. In specific, the device wearer 100 is looking at the accessory device 324. In this view, the accessory device 600 includes a display screen 1202 and a camera 1204.

The camera 1204 of the accessory device 600 can be focused on the device wearer 100 and can detect various visual aspects/features of the device wearer 100. To facilitate this, in some embodiments the ear-wearable device 120 is configured to prompt the device wearer 100 to look at the accessory device 600 (equipped with a camera 1204) if a pattern indicative of an occurrence of an anoxic or hypoxic neurological injury is detected.

Many different visual aspects/features are contemplated herein. In various embodiments, the ear-wearable device 120 can detect non-volitional eye movement by virtue of the camera 1204 capturing images of the device wearer 100. In some embodiments, the ear-wearable device 120 and/or a device in communication with the ear-wearable device can be configured to detect eye dilation through the use of data gathered with the camera 1204. In various embodiments, the ear-wearable device 120 and/or a device in communication with the ear-wearable device can be configured to detect facial paralysis, face droop or actions that may be consistent with drooling such as characteristic head movements associated with wiping of the device wearer's face.

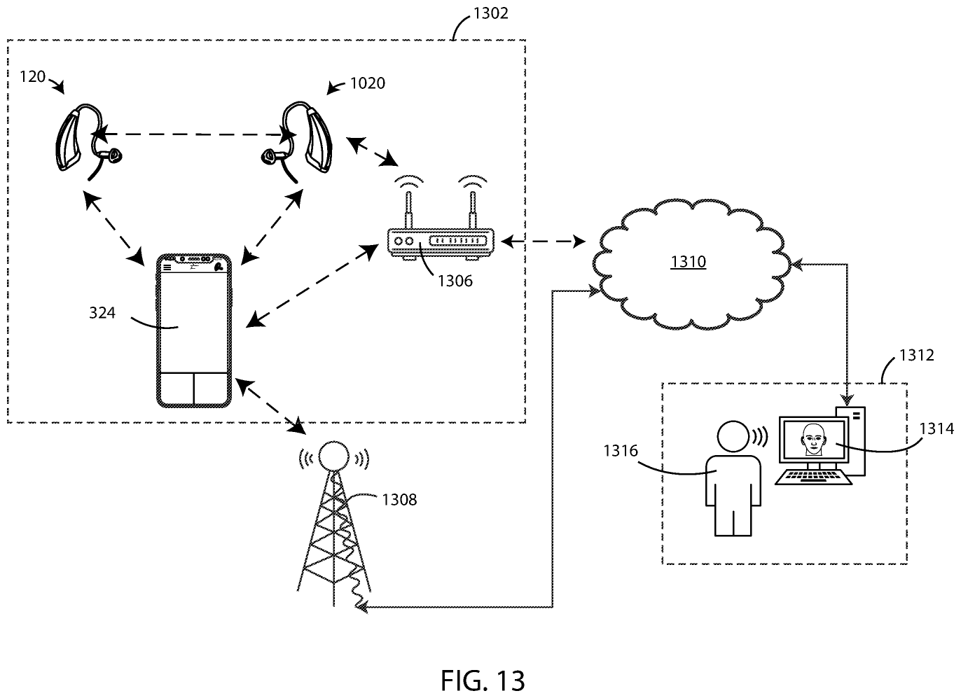

Referring now to FIG. 13, a schematic view is shown of data and/or signal flow as part of a system in accordance with various embodiments herein. In a first location 1302, a device wearer (not shown) can have a first ear-wearable device 120 and a second ear-wearable device 1020. Each of the ear-wearable devices 120, 1020 can include sensor packages as described herein including, for example, an IMU. The ear-wearable devices 120, 1020 and sensors therein can be disposed on opposing lateral sides of the subject's head. In some embodiments, the ear-wearable devices 120, 1020 and sensors therein can be disposed in a fixed position relative to the subject's head. The ear-wearable devices 120, 1020 and sensors therein can be disposed within opposing ear canals of the subject. The ear-wearable devices 120, 1020 and sensors therein can be disposed on or in opposing ears of the subject. The ear-wearable devices 120, 1020 and sensors therein can be spaced apart from one another by a distance of at least 3, 4, 5, 6, 8, 10, 12, 14, or 16 centimeters and less than 40, 30, 28, 26, 24, 22, 20 or 18 centimeters, or by a distance falling within a range between any of the foregoing.

In various embodiments, data and/or signals can be exchanged directly between the first ear-wearable device 120 and the second ear-wearable device 1020. An accessory device 600 (which could be an external visual display device with a video display screen, such as a smart phone amongst other things) can also be disposed within the first location 1302. The accessory device 600 can exchange data and/or signals with one or both of the first ear-wearable device 120 and the second ear-wearable device 1020 and/or with an accessory to the ear-wearable devices (e.g., a remote microphone, a remote control, a phone streamer, etc.). The accessory device 600 can also exchange data across a data network to the cloud 1310, such as through a wireless signal connecting with a local gateway device, such as a network router 1306, mesh network, or through a wireless signal connecting with a cell tower 1308 or similar communications tower. In some embodiments, the external visual display device can also connect to a data network to provide communication to the cloud 1310 through a direct wired connection.

In some embodiments, a care provider 1316 (such as an audiologist, speech-language pathologist, physical therapist, occupational therapist, a physician or a different type of clinician, specialist, or care provider) can receive information from devices at the first location 1302 remotely at a second location 1312 through a data communication network such as that represented by the cloud 1310. The care provider 1316 can use a computing device 1314 to see and interact with the information received. The computing device 1314 could be a computer, a tablet device, a smartphone, or the like. The received information can include, but is not limited to, information regarding the subject's response time (reaction time and/or reflex time). In some embodiments, received information can be provided to the care provider 1316 in real time. In some embodiments, received information can be stored and provided to the care provider 1316 at a time point after response times are measured.

In some embodiments, the care provider 1316 (such as an audiologist, physical therapist, a physician or a different type of clinician, specialist, or care provider, or physical trainer) can send information remotely from the second location 1312 through a data communication network such as that represented by the cloud 1310 to devices at the first location 1302. For example, the care provider 1316 can enter information into the computing device 1314, can use a camera connected to the computing device 1314 and/or can speak into the external computing device. The sent information can include, but is not limited to, feedback information, guidance information, therapy prescription, device programming related to therapy, and the like. In some embodiments, feedback information from the care provider 1316 can be provided to the subject in real time.

As such, embodiments herein can include operations of sending data to a remote system user at a remote site, receiving feedback from the remote system user, and presenting the feedback to the subject. The operation of presenting the auditory feedback to the subject can be performed with the ear-wearable device (s). In various embodiments, the operation of presenting the auditory feedback to the subject can be performed with an ear-wearable device(s).

Ear-wearable devices of the present disclosure can incorporate an antenna arrangement coupled to a high-frequency radio, such as a 2.4 GHz radio. The radio can conform to an IEEE 802.11 (e.g., WIFI®) or BLUETOOTH® (e.g., BLE, BLUETOOTH® 4.2 or 5.0) specification, for example. It is understood that ear-wearable devices of the present disclosure can employ other radios, such as a 900 MHz radio or radios operating at other frequencies or frequency bands. Ear-wearable devices of the present disclosure can be configured to receive streaming audio (e.g., digital audio data or files) from an electronic or digital source. Representative electronic/digital sources (also referred to herein as accessory devices) include an assistive listening system, a TV streamer, a radio, a smartphone, a cell phone/entertainment device (CPED) or other electronic device that serves as a source of digital audio data or files. Systems herein can also include these types of accessory devices as well as other types of devices.

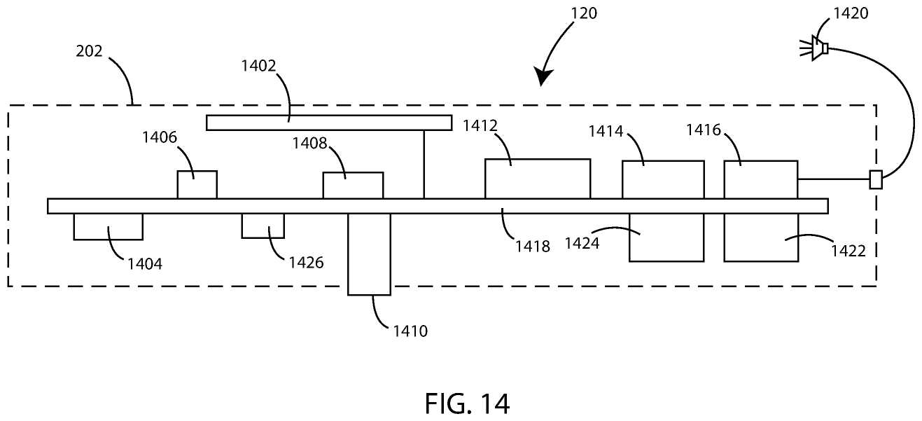

Referring now to FIG. 14, a schematic block diagram is shown with various components of an ear-wearable device in accordance with various embodiments. The block diagram of FIG. 14 represents a generic ear-wearable device for purposes of illustration. The ear-wearable device 120 shown in FIG. 14 includes several components electrically connected to a flexible mother circuit 1418 (e.g., flexible mother board) which is disposed within housing 302. A power supply circuit 1404 can include a battery and can be electrically connected to the flexible mother circuit 1418 and provides power to the various components of the ear-wearable device 120. One or more microphones 1406 are electrically connected to the flexible mother circuit 1418, which provides electrical communication between the microphones 1406 and a digital signal processor (DSP) 1412. Among other components, the DSP 1412 incorporates or is coupled to audio signal processing circuitry configured to implement various functions described herein. A sensor package 1414 can be coupled to the DSP 1412 via the flexible mother circuit 1418. The sensor package 1414 can include one or more different specific types of sensors such as those described in greater detail below. One or more user switches 1410 (e.g., on/off, volume, mic directional settings) are electrically coupled to the DSP 1412 via the flexible mother circuit 1418. It will be appreciated that the user switches 1410 can extend outside of the housing 302.

An audio output device 1416 is electrically connected to the DSP 1412 via the flexible mother circuit 1418. In some embodiments, the audio output device 1416 comprises a speaker (coupled to an amplifier). In other embodiments, the audio output device 1416 comprises an amplifier coupled to an external receiver 1420 adapted for positioning within an ear of a wearer. The external receiver 1420 can include an electroacoustic transducer, speaker, or loud speaker. The ear-wearable device 120 may incorporate a communication device 1408 coupled to the flexible mother circuit 1418 and to an antenna 1402 directly or indirectly via the flexible mother circuit 1418. The communication device 1408 can be a BLUETOOTH® transceiver, such as a BLE (BLUETOOTH® low energy) transceiver or other transceiver(s) (e.g., an IEEE 802.11 compliant device). The communication device 1408 can be configured to communicate with one or more external devices, such as those discussed previously, in accordance with various embodiments. In various embodiments, the communication device 1408 can be configured to communicate with an external visual display device such as a smart phone, a video display screen, a tablet, a computer, or the like.

In various embodiments, the ear-wearable device 120 can also include a control circuit 1422 and a memory storage device 1424. The control circuit 1422 can be in electrical communication with other components of the device. In some embodiments, a clock circuit 1426 can be in electrical communication with the control circuit. The control circuit 1422 can execute various operations, such as those described herein. The control circuit 1422 can include various components including, but not limited to, a microprocessor, a microcontroller, an FPGA (field-programmable gate array) processing device, an ASIC (application specific integrated circuit), or the like. The memory storage device 1424 can include both volatile and non-volatile memory. The memory storage device 1424 can include ROM, RAM, flash memory, EEPROM, SSD devices, NAND chips, and the like. The memory storage device 1424 can be used to store data from sensors as described herein and/or processed data generated using data from sensors as described herein.

It will be appreciated that various of the components described in FIG. 14 can be associated with separate devices and/or accessory devices to the ear-wearable device. By way of example, microphones can be associated with separate devices and/or accessory devices. Similarly, audio output devices can be associated with separate devices and/or accessory devices to the ear-wearable device. Further accessory devices as discussed herein can include various combinations of the components as described with respect to an ear-wearable device. For example, an accessory device can include a control circuit, a microphone, a motion sensor, and a power supply, amongst other things.

### Pattern Identification

It will be appreciated that in various embodiments herein, a device or a system can be used to detect a pattern or patterns (such as patterns of data from sensors) indicative of a state of recovery from an anoxic or hypoxic neurological injury as well as patterns relating to the same over time. Also, it will be appreciated that in various embodiments herein, a device or a system can be used to detect a pattern or patterns indicative of the execution of a rehabilitation therapy to determine whether it has occurred and/or how well a rehabilitation therapy or therapy step has been performed. Such patterns can be detected in various ways. Some techniques are described elsewhere herein, but some further examples will now be described.

As merely one example, one or more sensors can be operatively connected to a controller (such as the control circuit described in FIG. 14) or another processing resource (such as a processor of another device or a processing resource in the cloud). The controller or other processing resource can be adapted to receive data representative of a characteristic of the subject from one or more of the sensors and/or determine statistics of the subject over a monitoring time period based upon the data received from the sensor. As used herein, the term “data” can include a single datum or a plurality of data values or statistics. The term “statistics” can include any appropriate mathematical calculation or metric relative to data interpretation, e.g., probability, confidence interval, distribution, range, or the like. Further, as used herein, the term “monitoring time period” means a period of time over which characteristics of the subject are measured and statistics are determined. The monitoring time period can be any suitable length of time, e.g., 1 millisecond, 1 second, 10 seconds, 30 seconds, 1 minute, 10 minutes, 30 minutes, 1 hour, etc., or a range of time between any of the foregoing time periods.

Any suitable technique or techniques can be utilized to determine statistics for the various data from the sensors, e.g., direct statistical analyses of time series data from the sensors, differential statistics, comparisons to baseline or statistical models of similar data, etc. Such techniques can be general or individual-specific and represent long-term or short-term behavior. These techniques could include standard pattern classification methods such as Gaussian mixture models, clustering as well as Bayesian approaches, neural network models and deep learning.

Further, in some embodiments, the controller can be adapted to compare data, data features, and/or statistics against various other patterns, which could be prerecorded patterns (baseline patterns) of the particular individual wearing an ear-wearable device herein, prerecorded patterns (group baseline patterns) of a group of individuals wearing ear-wearable devices herein, one or more predetermined patterns that serve as positive example patterns (such as patterns indicative of functional state after an anoxic or hypoxic neurological injury or therapy performance), negative example patterns, or the like. As merely one scenario, if a pattern is detected in an individual that exhibits similarity crossing a threshold value to a positive example pattern or substantial similarity to that pattern, then that can be taken as an indication of the presence of a functional state associated with the positive example pattern. Positive and/or negative example patterns can be stored or accessed for use covering those items to be detected in accordance with embodiments herein including, but not limited to, therapy performance, therapy steps, examples of good therapy step performance, examples of bad therapy step performance, examples of specific levels of functional performance across domains such as motor function, speech function, neurological function, and the like.

Similarity and dissimilarity can be measured directly via standard statistical metrics such normalized Z-score, or similar multidimensional distance measures (e.g. Mahalanobis or Bhattacharyya distance metrics), or through similarities of modeled data and machine learning. These techniques can include standard pattern classification methods such as Gaussian mixture models, clustering as well as Bayesian approaches, neural network models, and deep learning.

As used herein the term “substantially similar” means that, upon comparison, the sensor data are congruent or have statistics fitting the same statistical model, each with an acceptable degree of confidence. The threshold for the acceptability of a confidence statistic may vary depending upon the subject, sensor, sensor arrangement, type of data, context, condition, etc.

The statistics associated with the health status of an individual (and, in particular, their status with respect to an anoxic or hypoxic neurological insult/injury), over the monitoring time period, can be determined by utilizing any suitable technique or techniques, e.g., standard pattern classification methods such as Gaussian mixture models, clustering, hidden Markov models, as well as Bayesian approaches, neural network models, and deep learning.

### Methods

Many different methods are contemplated herein. Aspects of system/device operation described elsewhere herein can be performed as operations of one or more methods in accordance with various embodiments herein.

### Methods

In an embodiment, a method of providing a therapy to an individual that has suffered an anoxic or hypoxic injury is included, the method initiating a therapy for the individual using an ear-wearable device, and monitoring signals from a microphone and/or a motion sensor of the ear-wearable device to detect execution of the therapy.

In an embodiment, the method can further include directing the individual to execute steps of the therapy using the ear-wearable device. In an embodiment, the method can further include directing the individual using the ear-wearable device by providing audible instructions.

In an embodiment, the method can further include evaluating a nature or quality of a response from the individual in response to the therapy. In an embodiment of the method, the nature or quality of the response includes at least one of fricative stopping, liquid gliding, lisping, dysphonia, and disfluency.

In an embodiment, the method can further include observing the speech of the individual during the course of therapy sessions. In an embodiment, the method can further include observing the speech of the individual outside of therapy sessions.

In an embodiment of the method, the therapy comprises speech therapy. In an embodiment of the method, the therapy comprises swallow therapy. In an embodiment of the method, the therapy comprises motor skills therapy. In an embodiment of the method, the therapy comprises cognitive therapy.

In an embodiment, the method can further include detecting head position, swallowing, and/or drinking during or after a therapy session.

In an embodiment, the method can further include detecting aspiration during or after a therapy session.

In an embodiment of the method, initiating the therapy is triggered based on at least one of detection of an acoustic environment, detection of motion, and the occurrence of a specific date and/or time.

In an embodiment, the method can further include providing an adaptive recommendation to the individual using the ear-wearable device.

In an embodiment, the method can further include tracking hydration of the individual using the ear-wearable device.

In an embodiment, the method can further include sending therapy instructions using the ear-wearable device to an accessory device for visual presentation to the individual.

In an embodiment, a method of monitoring recovery of an individual from an anoxic or hypoxic injury is included, the method recording signals from at least one of a microphone and a motion sensor of an ear-wearable device, and evaluating the recorded signals to assess recovery from an anoxic or hypoxic neurological injury.

In an embodiment, the method can further include querying the individual using the ear-wearable device. In an embodiment, the method can further include evaluating a nature or quality of a response from the individual in response to the query.

In an embodiment, the method can further include evaluating trends in at least one of posture, gait, sway, foot shuffling, stride symmetry, and foot fall intensity. In an embodiment, the method can further include evaluating trends in movement speed of the device wearer.

In an embodiment, the method can further include evaluating trends in movement patterns and/or activity levels of the device wearer.

In an embodiment, the method can further include evaluating signals from at least one of the microphone and the motion sensor to detect patterns indicative of sequelae of an anoxic or hypoxic neurological injury.

In an embodiment, the method can further include prompting the individual to look at an accessory device equipped with a camera.

### Sensors

Ear-wearable devices as well as medical devices herein can include one or more sensor packages (including one or more discrete or integrated sensors) to provide data. The sensor package can comprise one or a multiplicity of sensors. In some embodiments, the sensor packages can include one or more motion sensors (or movement sensors) amongst other types of sensors. Motion sensors herein can include inertial measurement units (IMU), accelerometers, gyroscopes, barometers, altimeters, and the like. The IMU can be of a type disclosed in commonly owned U.S. patent application Ser. No. 15/331,230, filed Oct. 21, 2016, which is incorporated herein by reference. In some embodiments, electromagnetic communication radios or electromagnetic field sensors (e.g., telecoil, NFMI, TMR, GMR, etc.) sensors may be used to detect motion or changes in position. In some embodiments, biometric sensors may be used to detect body motions or physical activity. Motions sensors can be used to track movement of a patient in accordance with various embodiments herein.

In some embodiments, the motion sensors can be disposed in a fixed position with respect to the head of a patient, such as worn on or near the head or ears. In some embodiments, the operatively connected motion sensors can be worn on or near another part of the body such as on a wrist, arm, or leg of the patient.

According to various embodiments, the sensor package can include one or more of an IMU, and accelerometer (3, 6, or 9 axis), a gyroscope, a barometer, an altimeter, a magnetometer, a magnetic sensor, an eye movement sensor, a pressure sensor, an acoustic sensor, a telecoil, a heart rate sensor, a global positioning system (GPS), a temperature sensor, a blood pressure sensor, an oxygen saturation sensor, an optical sensor, a blood glucose sensor (optical or otherwise), a galvanic skin response sensor, a cortisol level sensor (optical or otherwise), a microphone, acoustic sensor, an electrocardiogram (ECG) sensor, electroencephalography (EEG) sensor which can be a neurological sensor, eye movement sensor (e.g., electrooculogram (EOG) sensor), myographic potential electrode sensor (EMG), a heart rate monitor, a pulse oximeter or oxygen saturation sensor (SpO2), a wireless radio antenna, blood perfusion sensor, hydrometer, sweat sensor, cerumen sensor, air quality sensor, pupillometry sensor, cortisol level sensor, hematocrit sensor, light sensor, image sensor, and the like.

In some embodiments, the sensor package can be part of an ear-wearable device. However, in some embodiments, the sensor packages can include one or more additional sensors that are external to an ear-wearable device. For example, various of the sensors described above can be part of a wrist-worn or ankle-worn sensor package, or a sensor package supported by a chest strap. In some embodiments, sensors herein can be disposable sensors that are adhered to the device wearer (“adhesive sensors”) and that provide data to the ear-wearable device or another component of the system.

Data produced by the sensor(s) of the sensor package can be operated on by a processor of the device or system.

As used herein the term “inertial measurement unit” or “IMU” shall refer to an electronic device that can generate signals related to a body's specific force and/or angular rate. IMUs herein can include one or more accelerometers (3, 6, or 9 axis) to detect linear acceleration and a gyroscope to detect rotational rate. In some embodiments, an IMU can also include a magnetometer to detect a magnetic field.

An eye movement sensor herein can be, for example, an electrooculographic (EOG) sensor, such as an EOG sensor disclosed in commonly owned U.S. Pat. No. 9,167,356, which is incorporated herein by reference. The pressure sensor can be, for example, a MEMS-based pressure sensor, a piezo-resistive pressure sensor, a flexion sensor, a strain sensor, a diaphragm-type sensor and the like.

A temperature sensor herein can be, for example, a thermistor (thermally sensitive resistor), a resistance temperature detector, a thermocouple, a semiconductor-based sensor, an infrared sensor, or the like.

A blood pressure sensor herein can be, for example, a pressure sensor. The heart rate sensor can be, for example, an electrical signal sensor, an acoustic sensor, a pressure sensor, an infrared sensor, an optical sensor, or the like.

An oxygen saturation sensor (such as a blood oximetry sensor) herein can be, for example, an optical sensor, an infrared sensor, a visible light sensor, or the like.

An electrical signal sensor herein can include two or more electrodes and can include circuitry to sense and record electrical signals including sensed electrical potentials and the magnitude thereof (according to Ohm's law where V=IR) as well as measure impedance from an applied electrical potential.

It will be appreciated that the sensor package can include one or more sensors that are external to the ear-wearable device. In addition to the external sensors discussed hereinabove, the sensor package can comprise a network of body sensors (such as those listed above) that sense movement of a multiplicity of body parts (e.g., arms, legs, torso). In some embodiments, the ear-wearable device can be in electronic communication with the sensors or processor of another medical device, e.g., an insulin pump device or a heart pacemaker device.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein. Any of the methods or embodiments disclosed herein can be combined with any of the other methods or embodiments disclosed herein unless the context dictates otherwise.

1. An ear-wearable device comprising:
a control circuit;
a microphone, wherein the microphone is in electrical communication with the control circuit;
a motion sensor, wherein the motion sensor is in electrical communication with the control circuit; and
a power supply circuit, wherein the power supply circuit is in electrical communication with the control circuit;
wherein the ear-wearable device is configured to initiate a therapy for a wearer of the ear-wearable device for recovery from an anoxic or hypoxic neurological injury and monitor signals from the microphone and/or the motion sensor to detect execution of the therapy.

2-3. (canceled)

4. The ear-wearable device of claim 1, the therapy comprising speech-language therapy.

5. The ear-wearable device of claim 1, wherein the ear-wearable device is configured to evaluate a nature or quality of a response from the ear-wearable device wearer in response to the therapy, wherein the nature or quality of the response includes at least one of fricative stopping, liquid gliding, lisping, dysphonia, and disfluency.

6. (canceled)

7. The ear-wearable device of claim 1, wherein the ear-wearable device is configured to evaluate the ear-wearable device wearer's response to the therapy as observed by their speech outside of therapy sessions.

8. The ear-wearable device of claim 1, the therapy comprising swallow therapy, wherein the swallow therapy comprises a swallow protocol.

9. (canceled)

10. The ear-wearable device of claim 8, wherein the ear-wearable device is configured to use signals from the microphone and/or the motion sensor to detect head position, swallowing, and/or drinking during execution of the swallow protocol.

11. The ear-wearable device of claim 8, wherein the ear-wearable device is configured to use signals from the microphone and/or the motion sensor to detect aspiration during execution of the swallow protocol.

12. The ear-wearable device of claim 1, the therapy comprising motor skills therapy.

13. The ear-wearable device of claim 12, the motor skills therapy comprising a movement protocol.

14. The ear-wearable device of claim 12, the motor skills therapy comprising at least one of range of motion therapy, mobility training, limb movement, and virtual reality therapy.

15. The ear-wearable device of claim 1, the therapy comprising cognitive therapy.

16. The ear-wearable device of claim 1, wherein initiating the therapy is triggered based on at least one of detection of an acoustic environment, detection of motion, and an occurrence of a specific date and/or time.

17. The ear-wearable device of claim 1, wherein the ear-wearable device is configured to provide an adaptive recommendation, wherein the adaptive recommendation comprises liquid thickening.

18-20. (canceled)

21. The ear-wearable device of claim 1, wherein the ear-wearable device is configured to receive an input from the wearer of the ear-wearable device to delay, reschedule, or cancel the therapy.

22. An ear-wearable device comprising:
a control circuit;
a microphone, wherein the microphone is in electrical communication with the control circuit;
a motion sensor, wherein the motion sensor is in electrical communication with the control circuit; and
a power supply circuit, wherein the power supply circuit is in electrical communication with the control circuit;
wherein the ear-wearable device is configured to evaluate signals from at least one of the microphone and the motion sensor to assess recovery from an anoxic or hypoxic neurological injury.

23. The ear-wearable device of claim 22, wherein the ear-wearable device is configured to query the device wearer and evaluate a nature or quality of a response from the device wearer in response to the query.

24. (canceled)

25. The ear-wearable device of claim 22, wherein the ear-wearable device is configured to evaluate trends in at least one of posture, gait, sway, foot shuffling, stride symmetry, and foot fall intensity.

26. (canceled)

27. The ear-wearable device of claim 22, wherein the ear-wearable device is configured to evaluate trends in movement patterns and/or activity levels of the device wearer.

28. The ear-wearable device of claim 22, wherein the ear-wearable device is configured to evaluate signals from at least one of the microphone and the motion sensor to detect patterns indicative of sequelae of an anoxic or hypoxic neurological injury, the patterns comprising at least one of
changed pronunciation;
slurred words; and
breathiness, pitch change, vowel instability, and/or roughness of the ear-wearable device wearer's speech;
long delays; and
words indicating confusion.

29-38. (canceled)

39. The ear-wearable device of claim 22, wherein the ear-wearable device is configured to detect at least one of a non-volitional body movement and a non-volitional eye movement.

40-68. (canceled)
